(19) United States
(12) Patent Application Publication
(10) Pub. No.: US 2014/0275863 A1
(43) Pub. Date: Sep. 18, 2014

(54) FLUID ANALYSIS DEVICE AND RELATED METHOD

(71) Applicant: VITAL HERD, INC., Falmouth, ME (US)

(72) Inventors: Karim M. Gabriel, Lunenburg, MA (US); Brian F. Walsh, Falmouth, ME (US)

(73) Assignee: VITAL HERD, INC., Falmouth, ME (US)

(21) Appl. No.: 13/802,931

(22) Filed: Mar. 14, 2013

(51) Int. Cl. A61B 5/145; A61D 7/00

(52) U.S. Cl. CPC A61B 5/14507; A61D 7/00
USPC 600/309

(57) ABSTRACT

A device and method for analyzing characteristics of a fluid. The device is a bolus arranged to remain within the fluid for the analysis. The bolus includes a sensor module, a fluid analysis module, a fluid pumping module and a control and data transmission module. The sensor module includes a transducer element to generate acoustic signals and convert acoustic signals. The information derived from generating and receiving acoustic signals is used to obtain information about constituents and conditions of the fluid. That information may be transferred to a near or remote location for analysis. The device and analysis method are suitable for evaluating the health of animals. The device may be used in a telemedicine environment at an animal health facility. It may also be uploaded to a database for use in the analysis of a group of sources of fluid, such as a herd of cows.

### DESCRIPTION OF THE INVENTION

1. Field of the Invention

The subject invention relates to devices used to analyze and/or monitor the characteristics of fluids, including fluid mixtures. More particularly, the present invention relates to analyzing and/or monitoring over an extended period of time the characteristics of fluids located in places that are difficult to access. The invention has wide applicability ranging from industrial processes to monitoring the health of animals including, for example, ruminating animals such as cows and buffaloes.

2. Description of the Prior Art

A health and productivity monitoring rumen bolus is an electronic device inserted into the rumen or reticulum of a ruminant animal to monitor one or more physiological or physical parameters to detect subclinical symptoms of disease, evaluate production efficiency and provide a unique means of identification. Herein, the term rumen shall include the reticulum even if not specifically noted. They are often called intra-rumen bolus sensors and there are several versions of these types of sensing devices in existence. Most of the existing devices incorporate multiple sensors while some only provide a means of animal serialization. Some devices utilize a single sensor to provide limited information on the animal's well-being. In some versions, the sensors are disposed outside the bolus.

The U.N. estimates that food production must double by 2050 to feed the world's increasing population. 70% of this new demand must be produced on the industry's current land footprint, meaning that technology is needed to improve production efficiencies on dairy and beef operations. According to industry and USDA data, 40% of dairy production cows get sick (morbidity) and 8% die (mortality) each year while 20% of feedlot cows get sick and 3% die. Last year four million animals died in the U.S., with many more getting sick. The economic impact from these production inefficiencies far exceeds $5 billion each year in the U.S. alone from lost revenue, unrewarded expenses, feed costs, treatment costs and rendering fees.

These high morbidity and mortality rates for both dairy and beef production have been consistent for over a decade and experts argue that these percentages are rising as genetics breed higher producing but more disease-prone animals. The cause for these production inefficiencies stems from how dairy and feedlot productivity and health is managed. Animals are observed a couple times each day for thirty minutes or so to isolate animals showing physical symptoms of depression. At this point in an illness's progression, an animal is ‘clinical’ and very difficult to treat to achieve full recovery. For infectious diseases like Bovine Respiratory Disease (BRD), which is the top cause of death on feedlots today, any clinical animal showing observable, physical symptoms has already infected up to 40 of its herd mates, which, in turn, continue to infect others. This human labor-dependent, infrequent observation approach fails to catch issues in the subclinical stage where they can be more effectively managed and treated. This industry-wide practice of subjective, infrequent herd observation to monitor health and productivity is the fundamental cause for the industry's consistently high mortality and morbidity rates.

Intra-rumen health monitoring devices capture and make usable an animal's sub-clinical physiological, health and metabolic information, offering dairy farmers and feedlot operators an earlier and more frequent means of collecting information that is beneficial in optimizing their operations and is also noninvasive to the animal. This information also enables regulatory agencies to make timely, quality decisions and take regulatory actions to ensure proper regulatory compliance, monitor changes in feed and feed additives, minimize the transmission of zoonotic diseases, and increase the efficiency of production of animal-derived food.

These devices provide information on various health and metabolic parameters and the influence of changes to feed and feed additives on these parameters. In addition, they facilitate the generation of unique animal identification, enhancing biosecurity by creating a tamper proof, unalterable serialization that facilitates tracking animals throughout the production process. This capability can significantly aid in early detection of zoonotic diseases, such as Listeriosis and Salmonellosis. Some of these existing devices also provide the tools for detecting food-borne pathogens such as Leptospirosis and Brucellosis, which show no clinical signs except late term abortion.

Current intra-rumen analysis/monitoring devices utilize an outer shell, which houses and seals in the discrete sensor or sensors and electronics. This device design results in a product that cannot be tested until after final assembly when significant value has been added to the unit. This approach also leads to higher in-field failures since the seal is not always perfect and a leak in the housing destroys the entire sensor/electronics assembly. It also limits recycling and refurbishment since the methods used to establish a liquid-tight seal complicate the disassembly of the device. The combination of these challenges in current devices results in a high cost product with limited capabilities and, often times, high in-field failure rates.

The configuration and technology that facilitates the use of intra-rumen devices lends itself to remote monitoring of any fluid in a confined space where the fluid is difficult to access. The design of such devices allows for the placement in various environments with minimal impact to the environment. The wireless transmission and long battery life make it ideal for monitoring changes in fluid properties for extended periods with minimal maintenance. Unfortunately, the existing devices available to analyze and/or monitor fluids under a range of difficult-to-access locations have limitations that make them unreliable enough and costly enough to restrict their widespread acceptance.

### BRIEF SUMMARY OF THE INVENTION

The present invention solves the problems associated with existing devices used to analyze and/or monitor the characteristics of fluids located in confined spaces. The invention has wide applicability ranging from industrial processes to monitoring the health of animals including, for example, ruminating animals such as cows and buffaloes. The invention is a device and related method for most any desired analysis of monitoring of the characteristics of a fluid using at least ultrasonic spectroscopy. The invention provides a cheaper, more practical way of monitoring the composition of fluids in a variety of industries including, but not limited to, production and other animal health, waste water treatment, industrial processes, enhanced oil recovery, hydraulic fracturing, also referred to as “fracking.” The device includes a sensor that can be positioned into a container of fluid. It facilitates a process for gathering information on the contents of the container. In one embodiment, the invention may be used for monitoring bodies of fluid remotely for salinity, CO₂ content, acidity/alkalinity, and other characteristics that may be of interest. In the production animal industry, the device can be used to monitor the health and metabolism of ruminant animals such as cows and buffalos. While this disclosure describes that particular functionality, it is to be understood that it is not limited thereto.

The device of the present invention includes, in an embodiment suitable for monitoring the health of a ruminating animal, a bolus that utilizes a sensor, typically a piezoelectric material, to measure physiological parameters such as temperature, the pH of the rumen fluid, the concentration of constituents of the rumen fluid, heart rate, breathing rate, activity level, and the like. The bolus includes a sensor module, a fluid analysis module, a fluid pumping module and a control and data transmission module. Each module may be fabricated, sealed, and tested separately and then assembled into a final liquid-tight configuration. The modules and their components interact electrically with each other using an interconnect system that isolates the electrical signals from the fluid under analysis/monitoring. This bolus configuration allows for the recycling and refurbishment of the individual modules after use if that is of interest to a user.

A version of the sensor module includes a transducer formed of or including a piezoelectric material. The transducer may be used passively to convert acoustic information, including sounds within the animal, into an electrical signal. These signals are used to evaluate the general health and behavior of the animal. The transducer may also be used actively to generate an acoustic pulse, which may be used to measure rumen fluid pH, concentration of rumen fluid constituents, and rumen fluid temperature. The transducer may also include one or more layers of acoustic modification, which may be positioned near the piezoelectric material to reduce interface reflections at the piezoelectric material. One such layer may also or alternatively be shaped and used to act as a lens to shape the generated acoustic pulse and increase the sensitivity of the transducer. The piezoelectric material may also or alternatively be shaped and used to act as a lens to shape the generated acoustic pulse and increase the sensitivity of the transducer. The sensor module is liquid tight with two leads extending beyond the sealed module for connection with the fluid analysis module.

In one embodiment, the fluid analysis module admits fluid (a body fluid in the case of the device's use in monitoring animal health) into a chamber thereof in order to determine the temperature, pH level, concentration of fluid constituents, and other fluid properties. It is noted that in other embodiments, the fluid analysis module could admit water, wastewater, industrial fluids or any other fluid of interest that is difficult to access. The fluid analysis module may house a liquid-tight interconnect configuration that allows electrical communication between the sensor module and the control and data transmission module. The interconnect scheme facilitates high-volume, low-cost manufacturing methods. The chamber may also include a material with an acoustic impedance characteristic that is mismatched with respect to the acoustic impedance of the fluid under analysis, creating a reflective surface spaced from the transducer.

In one embodiment, the fluid pumping module may be configured to generate fluid flow into and out of the module and to remove any trapped gas that might negatively impact fluid analysis. It also may house a magnet that can be used to generate power and a spring or similar element to generate axial motion as a function of compression and extension of the spring.

The control and data transmission module, in one embodiment, includes three discrete sub modules: a power cell, a data processing and control sub-module, and a data transmission sub-module that, when assembled, establish a functional sub-element of the bolus. The sub modules of the control and data transmission module may be sealed to reduce fluid contact with its electrical components. The module has two electrical leads protruding from a liquid-tight assembly housing, which connects to an interconnector of the fluid analysis chamber, thereby enabling interaction with the sensor module without electronics degradation due to fluid exposure.

In one embodiment, the power cell sub module powers components of the control and data transmission module as well as the sensor module. In one embodiment of the control and data transmission module, a charging circuit of the data processing and control sub module stores voltage generated by the transducer of the sensor module in response to vibrations received by the transducer or as a result of the electric fields induced by the motion of the magnets on the fluid pumping module. The charging circuit is configured to supply this transducer-generated voltage to the power source to extend the life of the power cell and reduce the size and cost of this component.

In an embodiment of the control and data transmission module, the data processing and control sub module is configured to conduct on-board diagnosis of the subject animal. In this embodiment, the control sub module is configured to activate the transducer to provide a signal through the body fluid and to receive a signal generated by the transducer in response to vibrations. A microprocessor of the control sub module can be configured to measure the temperature of the module and thus the temperature of the subject animal under evaluation. Alternatively, a discrete temperature sensor, such as a thermistor, may be used with associated signal conditioning electronics, or an integrated circuit configured to measure temperature directly may be used to transmit signals to the microprocessor indicative of temperature characteristics.

In an embodiment of the control and data transmission module, the data processing and control sub module is configured to utilize the received signal from the pulse generated by the transducer to determine the concentration of constituents in, and/or the density of, the rumen fluid. This information allows for the calculation of the pH of the rumen fluid as well as the detection of increases in concentration of the constituents. It is noted that in other embodiments, the data processing and control sub module is configured to utilize the received signal from the generated pulse to determine the concentration of the various constituents and the density of other types of fluids including, for example, water, wastewater, industrial fluids or any other fluid used in a variety of industries, e.g., waste water treatment, industrial processes, enhanced oil recovery and hydraulic fracturing, but not limited thereto. The device may rely on calibration curves or various analytical, numerical or statistical models for predicting pH for determining the various constituents. Such information may be stored on the bolus or accessed in a location remote from the bolus.

The data transmission sub module optionally includes a transceiver that transmits data and stores in memory data and/or commands received from an external source. The data transmission sub module accomplishes signal exchanges with external mechanisms with an antenna that may be internal or external to the bolus.

In an embodiment of the invention, the bolus, through the data transmission sub module, transmits data to a retransmitting module of the invention located external to the bolus, which, in turn, transmits the data to a base station for further analysis on-site or remotely. The retransmitting module may be stationary or traverse an area in which the animals are contained. In the embodiment where the retransmitting module traverses the containment area, this may be accomplished via a wheeled or tracked or otherwise mobile platform. In this embodiment, the retransmitting module may be configured to, or be located in a device, such as a robotic device, that is configured to travel along one or more fixed paths that may be established by wires or tracks according to preprogrammed algorithms for speed, coverage, obstacle avoidance, etc. In this way, the retransmitting module and base station system utilizes fewer modules or base stations because they are mobile, covering more area than if they were stationary. In one embodiment, the retransmitting module or the base station may be powered via solar panels, one or more wind turbines, or other means of renewable energy.

### BRIEF DESCRIPTION OF THE DRAWINGS

FIG. 1 is a cross-sectional view of the fully assembled bolus of the present invention.

FIG. 2A is an exploded view of the bolus comprising the sensor module, the fluid analysis chamber and the control and data transmission module.

FIG. 2B is close up of a connector arrangement of the bolus.

FIG. 3A is a cross-sectional side view of the sensor module with a close-up of an example snap-fit component to secure parts of the module together.

FIG. 3B is an exploded view of the sensor module.

FIG. 3C is a close up of a connector arrangement of the bolus.

FIG. 3D is a cross sectional side view of a transducer element of the present invention.

FIG. 4 is a cross-sectional view of an embodiment of the acoustic lens of the sensor module and its function.

FIG. 5 is a graph showing a drive pulse for the transducer of the sensor module.

FIG. 6 is a cross-sectional view of a second embodiment of the transducer element.

FIG. 7A is a cross-sectional view of the fluid analysis chamber of the bolus.

FIG. 7B is a close-up of an example snap-fit component to secure module parts together.

FIG. 7C is a cross-sectional side view of a snap-fit arrangement that allows for slight misalignment of conductor elements.

FIG. 8 is a simplified representation of the control and data transmission module of the present invention.

FIG. 9 is a simplified representation of the retransmitter of the present invention.

FIG. 10 is a is a simplified representation of the base station of the present invention graph showing a time of flight calculation for a received pulse from the sensor module.

FIG. 11 is a simplified representation of a radio triangulation concept associated with transmitting signals from the bolus.

FIG. 12 is a flow chart showing steps of the method for analyzing signals from the sensor module.

FIG. 13 is a graph showing a received pulse from the sensor module.

FIG. 14 is a graph showing a time of flight calculation for a received pulse from the sensor module.

FIG. 15 is a graph showing the frequency spectrum of a 2.25 MHz immersion transducer.

FIG. 16 is a graph showing the frequency spectrum of acetic acid at 5.2 mL/L.

FIG. 17 is a graph showing the frequency spectrum of acetic acid at 3.2 mL/L.

FIG. 18 is a graph showing a baseline spectrum for acetic acid subtracting the spectra of FIGS. 16 and 17 with secondary processing including curve fitting, smoothing, and filtering.

FIG. 19 is a calibration curve for acetic acid based on a change in spectral response vs. concentration.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction, the analysis methods, and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

FIG. 1 shows an example of a bolus 10 in accordance with the invention, which is typically placed inside the rumen of an animal, (e.g., a cow) to monitor the health or productivity of the animal. It may also be used in another type of fluid-filled environment to monitor the properties and constituents of the fluid. In veterinary medicine, a bolus is a large time-release tablet that stays in the rumen of ruminant animals such as cattle, goats and sheep. In the context of this invention the term bolus is used to define an electromechanical device contained in a container arranged to withstand the environment within which it is placed. The container may be tablet-shaped in describing the present invention when used to stay in the rumen. It may be of the same shape or size or a different shape or size when used in other fluid-filled environments.

The bolus 10 in FIG. 1. is divided into four sections: a sensor module 11, a fluid analysis module 15, a fluid pumping module 19 and a control and data transmission module 18. Each module is fabricated, sealed as required and tested separately and then assembled into the final liquid tight configuration of the bolus 10. The various components interact electrically with each other using an interconnect system that is isolated from the fluid. This system configuration allows for the recycling and refurbishment of the individual modules after use. A spring 23 (to be described further herein) of the pumping module 19 of the bolus 10 may be compressed with a temporary bolus retainer of the type familiar to those skilled in the art of inserting boluses into ruminating animals and the pumping module 19 held in place by a temporary pump retaining attachment feature shown as elements 13a and 13b in FIG. 1. The elements 13a and 13b dissolve in the rumen to release the pump module 19, in a way that allows the fluid analysis module 15 to move axially with respect to the pump module facilitating drawing of fluid into the bolus 10. Features 13a and 13b represent what may be a ring that extends around the circumference of fluid analysis module 15, a split ring, or a plurality of discrete pieces. The temporary pump retaining features 13a and 13b may also be displaced in a way that permits the pumping activity described herein by the natural motion of the rumen or even as a result of the pressure supplied by the spring 23. The bolus 10 may be installed in the location of interest using a balling gun, which balling gun compresses the bolus 10 in a way that compresses the spring 23 and therefore eliminates the need to include features 13a and 13b. A balling gun may also be used to remove the temporary pump retaining feature 13a and 13b that keeps the bolus 10 in the compressed state such as by clipping or displacing the features 13a and 13b.

The sensor module 11 and the control and data transmission module 18 are sealed to prevent entry of the rumen fluid therein and are fitted with mechanical interfaces (e.g., threads, snap fits, etc.) to facilitate assembly and integration with each other using high volume, low cost manufacturing methods. The sensor module 11 and the control and data transmission module 18 are connected electrically through an interconnect method contained in the fluid analysis module 15 discussed below.

FIGS. 1 and 2A represent an assembly drawing and an exploded view drawing of the monitoring bolus 10 demonstrating how the individual components fit together providing both a reliable electrical connection and a liquid tight assembly. In one embodiment, a recess 25-1, as shown in FIG. 2B, may be included at the interface of each module to allow for the application of a sealing compound. The recess 25-1 increases the bonding area as well as creating a tortuous path for any fluid that may travel between the interface of the fluid analysis module 15 and the sealing compound. Features 290a and 290b described herein provide a way to correct for misalignment between the fluid analysis module 15 and the control and data transmission module 18.

The fluid analysis module 15 and the fluid pumping module 19 work in concert to prevent entry of particulate matter via mesh screens (not shown) located at a plurality of fluid entry ports 12a and 12b in to the module 15, transport fluid in and out of the module 15, and provide a substrate for magnets shown in FIG. 2 as 22a, 22b, 22c, and 22d and facilitates axial motion of magnets 22a, 22b, 22c, and 22d. The fluid analysis module 15 provides the radial, angular and axial location of the sensor module 11 with respect to a reflective target such as the control and data transmission module 18 or a metal substrate 101 placed at the bottom of 15 and a connector 26-1 to connect the sensor module 11 and the data and control and data transmission module 18 to the fluid analysis module 15 at 25a, 25b, 25c, and 25d. The fluid pumping section 19 facilitates motion of fluid under analysis/monitoring into and out of the fluid analysis section and is concentric with the inner section. Both of modules 15 and 19 can be molded, cast or fabricated by standard manufacturing processes and assembled using high volume, low cost manufacturing processes.

As shown in FIG. 2A, the fluid pumping module 19 includes a pump housing 21. The pump housing 21 may be made of plastic, metal, a composition or the like suitable for retaining one or more components of the bolus 10 therein and/or thereon. The fluid pump module 19 also includes a plurality of magnets 22a 22b, 22c and 22d extending about an exterior of the pump housing 21 in magnet retaining slots 29 thereof. The pumping section 19 also includes the spring 23 or a similar device suitable to provide a loading force capable of resisting or enabling axial movement of the fluid pumping module 19. A sealing component 25a and 25b and a mechanical feature such as connector 26-1 (e.g., snap fits, etc.), as shown in FIG. 2B, allow for alignment and retention of the pump housing 21 when mated to the inner section 15 of the fluid analysis module 15. The fluid pumping module 19, when integrated with the fluid analysis module 15, forms a seal that allows the pumping module 19 to draw fluid into the fluid analysis module 15 when the pump module 19 traverses axially away, which is caused by movement of the spring 23 from the sensor module 11. When the pumping module 19 traverses axially towards the sensor module 11, the fluid is forced out of the fluid analysis module 15.

The spring 23 of the pumping module 19 rests against a mechanical stop 28 located on the base of the fluid analysis module 15. The mechanical stop 28 also prevents the fluid analysis module 15 from sliding past the fluid pumping module 19 by impacting 24 located at the top of 19. The spring 23 functions to extend the fluid analysis module 15 when it is compressed due to naturally occurring rumen contractions and motion. The magnets 22a-22d function to: a) capture stray metal ingested by the animal; b) act as a counter weight to facilitate motion of the pumping module 19; and c) generate additional power by interacting with components in the control and data transmission module 18 to harvest the current induced by the axial motion of the magnets 22a-22d.

The fluid analysis module 15 can be made of plastic or other suitable material. The fluid analysis module 15 has mechanical features that allow for the location and attachment of 25a, 25b 25c, and 25d of the sensor module 11 and the control and data transmission module 18 as well as a mechanical feature 28 that acts as a stop for the spring 23 located in the pumping module 19. The fluid analysis module 15 also contains the electrical interconnects that electrically connect the sensor module 11 and the control and data transmission module 18 to each other, while providing a liquid tight seal around the electrical conductors. That interconnection arrangement is described more fully with respect to FIG. 7B.

FIG. 3A represent an assembly drawing of the sensor module 11 which includes a piezoelectric element 31 (e.g., a polycrystalline, single crystal, polymer, composite or other piezoelectric element) and, a sensor module housing 32, which contains components therein that may be sealed in, such as with a potting compound (not shown). The module 11 further includes a plurality of interface layers 34a and 34b adjacent to and sandwiching the piezoelectric element 31, and acoustic lenses 33a and 33b on either side of the interfaces 34a and 34b. The interface layers 34a and 34b are typically set to ¼ of the wavelength of the center frequency of the piezoelectric element 31 based on the speed of sound in the interface layer material. These interface layers serve to reduce the reflections due to acoustic impedance mismatches between layers. The acoustic impedance (Z) is equal to the velocity times the density of the material which the sound is passing through. To determine the desired impedance of the interface layer material, the square root of the product of the acoustic impedance of the two materials (the one the sound is leaving and the one the sound is entering) is used. This value becomes the desired acoustic impedance of the interface layer. Filled plastics such as Aluminum Nitride filled epoxies may be used where the concentration of the filler is determined by the change to both the density and velocity of the matrix material. Homogeneous materials such as graphite can be used when the impedance of the material meets the desired interface layer acoustic impedance. The sensor module 11 transmits an acoustic signal, when energized, to travel through the fluid in the fluid analysis module 15 and receives this signal, which can be used for measuring the pH level and/or temperature of the fluid in the fluid analysis module 15. The combination of the piezoelectric element 31, the interface layers 34a and 34b and the acoustic lenses 33a and 33b forms a transducer 311 of the present invention shown in FIG. 3D that is used to convert sound into electrical signals and to convert electrical signals into active sound pulses. An alternative transducer configuration is described with respect to FIG. 6.

The sensor module housing 32 has features 39a and 39b to facilitate the alignment and positioning of the piezoelectric element 31 allowing for consistent placement of the piezoelectric element 31 radially and axially in the bolus 10. This enables automated electrical attachment (e.g. soldering, conductive adhesive, thin non-conductive bonding, etc.) to the piezoelectric element 31 and high volume, low cost manufacturing (e.g. pick and place, etc.). Passing through the length of the housing 32 are two electrical conductors 35a and 35b, which may be molded in place or inserted after fabrication of the housing 32. These conductors 35a and 35b protrude out of a base 39a and a top 39b of the housing 32. The conductor 35a/35b that protrudes through the top 39a can be attached as previously described to the piezoelectric element 31. The connections can be made on the same face of the piezoelectric element 31 when the conductors 35a/35b are positioned to allow for same side attachment (i.e., the conductors 35a/35b are isolated). In instances where the conductors 35a/35b are configured for opposite side attachment, an additional feature such as a through hole is added to allow addressing of the interior one of the two conductors 35a/35b when soldering is used. This feature may be back filled to ensure a liquid tight seal. In instances where conductive adhesives are utilized, no such sealing feature may be required.

The sensor module housing 32 also includes assembly features 37a and 37b (e.g., threads, snap fits, etc.) which mate with the fluid analysis module 15 to create a liquid tight seal. These features also provide alignment between matting interconnect elements in the sensor module 11 and the fluid analysis chamber 18. These features allow for automated assembly and electrical attachment processes.

Once the fluid analysis module 15 is sealed, the sensor module 11 and the control and data transmission module 18 can be integrated and joined together using automated manufacturing processes. In the instances when the mechanical alignment elements are threaded elements, the sensor module 11 and control and data transmission module 18 may have a male threading 38-1 with a male snap fit feature 38-2 that allows for locking the modules in place to prevent unthreading. A representation of that is shown in FIG. 3C, which is a mating feature on the sensor housing 32. These or similar joining and/or alignment features may also be present on the control and data transmission module 18.

FIG. 4 shows the design of an example acoustic lens 44 suitable for use as part of the transducer element 311 of the present invention, which transducer element 311 is shown in FIGS. 3A-3D. It may have a logarithmic profile or other profile for focusing an acoustic beam 46. The acoustic lens 44 is applied to an outward radiating face 42 of the piezoelectric element 31, adjacent to an acoustic matching layer 43. The combination of the piezoelectric element 31, the lens 44 and the acoustic matching layer 43 constitute the transducer element 311 of the sensor module 11. The purpose of the lens 44 is to focus a projected acoustic beam 46 along axis 45 resulting in an increased sensitivity of the transducer element 311. This increase in sensitivity results in increased strength of the received signal. The power associated with that increased strength may be equivalent to the power of a preamplifier. A preamplifier may be used as part of one or more electronic components of the present invention as described herein. A reduction in the power of the preamplifier may extend the life of a battery that may be used to power one or more components of the bolus 10. The lens 44 can be shaped via molding of a material suitable for that purpose, such as an epoxy, filled epoxy, or similar material or machining a block of such material to a prescribed pattern. The lens 44 can be formed then attached to the piezoelectric element 42 or it can be formed in place. The piezoelectric element 42 can also be formed to include the acoustic lens 44. The acoustic lens 44 focuses the projected beam 46 at a reflective target 47, which is typically a material with an acoustic impedance substantially different from the medium in which the sound traverses 48, such as the fluid in the rumen of a cow, for example. Examples of such materials suitable to be the reflective target 47 include ceramics, metals, aerogels, etc. The transducer element's sensitivity is affected by the diameter of the projected beam 46 at the point of interest. Smaller beam diameters result in increased reflections at the interface 47. This increased signal allows for less power to be supplied to the piezoelectric element 31, resulting in decreased power consumption. The reduced power requirements can either extend battery life or reduce the total storage capacity required for the components of the modules of the bolus 10. In a passive mode, vibrations caused by breathing, motion, heartbeats, vocalizations, and the like are received as generally shown at the transducer element 311 and cause it to generate a signal indicative of these physiological or other parameters. The received signal, when not being analyzed, can be used to power an energy storage cell that can be used to help power components of the bolus 10.

The piezoelectric element 31 of the sensor module 11 can be used to generate an acoustic pulse, an example form of which is shown in FIG. 5. This represents the active mode for the sensor module 11. The active mode sends an acoustic pulse both into the fluid analysis module 15 and outward in to the body of the host animal. When the piezoelectric element 31 is energized, it might measure other physiological parameters such as body fat content and amount of muscle tissue as a result of the changes in frequency or velocity of the return signals (echoes) from the various body tissues that the sound passes through as it travels away from the exterior face of the transducer element 311. Alternately, in the passive mode, the changes in frequency and amplitude of internally or externally generated acoustic signals may be used to make predictions on the state of the animal. Thus, in one preferred embodiment, only one sensor is required to determine a number of physiological parameters.

FIG. 6 shows an alternate embodiment of a transducer element 60 in place of the transducer element 311 of FIG. 3. The transducer element 60 includes two piezoelectric elements 61a and 61b separated by an absorbing backing layer 62 of the sensor module 11. This configuration includes acoustic matching layers 63a and 63b and acoustic lenses 64a and 64b. The absorbing backing layer 62 includes a polymer filled with a high density material (e.g. Tungsten, a ceramic, etc.) and/or an absorbing material (e.g., rubber particulate). The polymer may be a homogeneous material or a heterogeneous material that includes discrete phases of absorbing material (e.g., rubber toughened epoxy, etc.) The advantage of this configuration is that it mechanically decouples the passive mode measurements from the active mode measurements and lends itself to simpler post processing analysis of the signal. Other materials may be used for the backing layer 62 provided they have similar decoupling characteristics.

FIG. 7A shows the fluid analysis module 15 of the bolus 10. The fluid analysis module 15, which may be cylindrical in shape and may be made of plastic, metal, composition, or the like suitable for retaining one or more components of the bolus 10 therein and/or thereon. The fluid analysis module 15 includes a module housing 71 that further includes mechanical locking and positioning elements 72a, 72b, 72c and 72d of the type previously described with respect to other mechanical locking and positioning elements of the bolus 10 at each end that allows for the mating of the other modules 11, 18 and 19 to the fluid analysis module 15. Passing through the radius/thickness of the module 15, along its axis, are electrical conductors 73a and 73b, e.g., wires, conductive molded plastic, metal coatings, etc. These conductors 73a and 73b electrically and mechanically connect the sensor module 11 to the control and data transmission module 19.

The fluid analysis module 15 can be produced in two sections and joined in a variety of means or as a single unit. The two sections can be connected in a clam shell configuration that folds together or exist as two discrete sections. The fluid analysis module 15 may have interlocking mechanical features (not shown) that facilitate the alignment and sealing of the housing 71 and may include a secondary sealant to provide a liquid tight seal.

The fluid analysis module 15 may be cast, molded, machined or fabricated in a manner suitable for high volume, low cost manufacturing. The electrical conductors 73a and 73b may be inserted into the housing 71 after fabrication or molded in place by suitable means and then the assembled and sealed to create a liquid tight unit.

Once the fluid analysis module 15 is sealed, the sensor module 11 and the control and data transmission module 18 can be integrated and joined together using automated manufacturing processes. One or more of the joining and/or alignment features, previously described, may be present on each of the modules. This joining of two or more of the modules of the bolus 10 may also be accomplished via the use of a sealing compound. For the mechanical joining configuration, the housing 71 of the fluid analysis module 15 includes a female threading with a female snap feature 72-2 and a recess to accept the male snap fit feature 724 as shown in FIG. 7B. The thread length, location of snap fit features, and start of thread are designed to ensure alignment of the conductors 73a and 73b when the threads are fully engaged. As shown in FIG. 7C, the conductor of the fluid analysis module 15 is designed to allow for slight misalignment of the electrical conductors 73a and 73b, represented in the figure as misalignment 78-1. Specifically, the electrical conductors 73a and 73b are bent as shown in FIG. 7C such that they overlap with their mating electrode. If a slight misalignment occurs, the electrode will still be in contact. The conductors 73a and 73b are bent at a 90 degree angle and rest in a recess (not shown) in housing 32 that is about the depth of the conductor thickness and is concentric with the circumference of 32. This feature is replicated on the sensor module 11 (see 36a and 36b) and the control and data transmission module 18. Electrically conductive joining material (e.g., solder, adhesive, etc.) can be applied prior to assembly of the modules or after. In instances when the material is applied after assembly, a recess as previously described is provided to provide access to the conductors 73a and 73b.

FIG. 8. shows the control and data transmission module 18, which includes a data processing sub module 172 and control circuitry 173 with related charging circuitry 174, which generates a signal to activate the transducer element 311 in the active mode and is responsive to, and detects, signals generated by the transducer element 311 in the passive mode. Such signals may include vibration signals indicative of movement, breathing, heart rate, and the like, as well as the reflection of the emitted acoustic signals generated during operation in the active mode off of various body tissue in the animal.

The data processing and control sub modules 172 and 173 also include a data analysis sub module 175 responsive to the control circuitry 173 for processing data such as comparing measured responses to a stored template derived from the various signals and/or conditioning the data via filtering, smoothing, compressing, and the like to facilitate transmission and reduce upstream processing. Memory 176 may also be provided to store these data, and condition and disease templates or analysis programs as well as a unique identifier (e.g. serial number). Antenna 180 and receiver/transmitter (transceiver) section 181 are used to transmit these data and/or some analysis of it, such as the partial or fully diagnosed condition or disease, as well as, optionally, a unique identifier, to outside of the bolus 10. Transmission will typically use RF frequencies and can also function to receive commands and data to modify the operation of the bolus 10.

The control and data transmission control module 18 further includes charging circuitry 174 that includes a power source 177, such as a battery, a capacitor or a hybrid system, to provide power to a transceiver 181 of the control and data transmission module 18 and the control circuitry 173. In one embodiment of the invention, the charging circuitry 174 is responsive to voltages generated by the transducer element 311 and these voltages are used to charge the power source 177. Voltages are generated by the transducer element 311, for example, in the passive mode, in response to vibrations. The result is that the bolus 10 has an energy harvesting subsystem. The charging circuitry 174 may also contain a coil conductor (not shown) such as copper wire that facilitates the generation of electricity as a result of current generated by linear induction resulting from the motion of the magnets 22a-22d located on the exterior of the pump housing 21.

The control circuitry 173 provides a charge signal, a pulse signal, or it receives a signal from the transducer element 311 when the transducer element 311 is in a passive mode. A pulse signal activates a transmit/receive switch 179, which may be amplified by an amplifier 171 as required, and then activates the transducer element 311 in the active mode to emit an acoustic signal.

Signals detected by the transducer element 311 in the passive mode pass through the transmit/receive switch 179, which determines if the charging circuit 174 is sending an electrical signal to the transducer element 311 or receiving an electrical signal from the transducer element 311. The received signal may be amplified by the amplifier 171 before or after the switch 179. The control circuitry 173 determines, for example, the time it takes for a signal to traverse the fluid in the fluid analysis module 15, reflect off the wall and traverse the fluid in the fluid analysis module 15 again before activating the transducer element 311. The control circuitry 173 may also determine, for example, the signal characteristics after reflection off body tissue or the signal characteristics after received by the transducer element 311 in the passive mode.

The data processing sub module 172 determines the type of data characterized by the signals received from the transducer element 311. The data processing, data analysis and control sub modules 172 and 175, and control circuitry 173, then process the signals to determine temperature, pH level, heart rate, respiration rate, motion, vocalizations, body fat content, digestive activity and the like. Typically, a microprocessor, application specific integrated circuit, controller, or the like is used to implement this functionality, wherein such a processing device implements instructions embodied in computer-executable software or firmware. Data analysis sub module 175 also preferably determines, based on these physiological parameters, one or more possible conditions of the subject and stores data indicating the same in the memory 176. This partially or fully diagnosed data is then transmitted. The data analysis sub module 175 may also send the raw data for independent analysis.

The data analysis sub module 175 then signals the transceiver sub module 181 of the control and data transmission module 18 to transmit this diagnosis via an antenna 180. The antenna 180 may be a separate discrete element or included as part of a signal transmission circuit 181. The data analysis sub module 175 may be configured (e.g. programmed) to activate transceiver 181 to send transmissions only if certain selectable conditions are characterized and then the transmission may occur on a selectable time basis, including ad hoc, sporadically or periodically. A signal receiver spaced away from the bolus 10, such as a base station 186 and/or a retransmitter 185 (discussed below with respect to FIGS. 9 and 10), then receives information generated by the control and data transmission module 18 regarding an identified condition or partial or full diagnosis information along with an identification of the bolus 10 that generated the information of interest. Personnel monitoring locally or remotely for such information can then take the appropriate corrective action or actions. The control circuitry 173 is also capable of receiving information and commands, via the transceiver 181 and antenna 180, allowing for updates to the templates and changes in the operational parameters housed in the data processing and control sub module 18.

The control circuitry 173 is configured to, at predetermined or selectable intervals, or at the direction of the base station 186, pulse the transducer element 311 to generate an acoustic signal for analysis. These intervals, or duty cycles, are based on optimizing the balance between power management and generating enough readings to provide sufficient fidelity to accurately characterize the health of the animal. Adjusting the duty cycle, using either on-board algorithms or by direction from the base station, extends the life of the power cell and allows for more intensive investigation of a particular animal or environment, as required. The received acoustic signal, when converted to an electrical signal by the sensor module 11, may be processed by the bolus 10 or sent to the base station for processing.

The signal including information of interest or to be processed to be transmitted by the bolus 10 may be transmitted by the transceiver 181 either to the retransmitter module 185 or the base station 186 described herein. The transceiver 181 is configured to broadcast a signal to the retransmitter module 185 to determine the readiness of such retransmitter module 185 to accept a subsequent transmission of information including data acquired in the course of processing the fluid. When the retransmitter module 185 is ready, i.e., not communicating with a bolus of a different location and is in range of the bolus 10, it will begin transmission of its data. The bolus 10 is configured to store transmittable data until such time as the transmission is possible; that is, that there exists a suitable recipient of such data including, for example, base station 186 or retransmitter module 185 ready and capable of receiving the transmission.

The interaction between the bolus 10 and the retransmitter module 185 can be used to locate the animal associated with that particular bolus 10 as required. Multiple retransmitters can be used to triangulate the bolus 10. When a disease condition is identified, or when the animal needs to be located or data from it simply gathered, a signal is sent to the bolus 10 to begin transmission of a location signal. This signal may be activated at the base station or locally by an operator communicating with the base station to start transmission. The position of the animal can be determined by measuring either the radial distance, or the direction, of the received signal from the bolus and two or more different retransmitters.

The bolus 10 may also interact with adjacent devices to create an ad hoc network. This network may be used to retransmit data when a particular device is not in range of the retransmitter module 185. The bolus 10 may also interact with other devices to perform analysis such as using the temperature output or other health information from adjacent devices to generate a profile of the herd temperature at a particular location or other health conditions.

The retransmitter module 185 shown in FIG. 9 may be equipped with a directional antenna 184, an omnidirectional antenna 182 or both in order to facilitate radio triangulation in addition to a dedicated antenna 183 to transmit data. FIG. 11 shows a representative example of the use of radio triangulation to locate a particular bolus. Multiple retransmission modules 161a, 161b, and 161c, which may be configured as shown are capable of receiving signals in an omnidirectional pattern, 163a, 163b, and 163c. The bolus 10 transmits its data signal to all retransmission modules 161a-161c. The strength of the signal is used to determine the distance 164a and 164b from the retransmission modules 161a-161c to the bolus 10. Combining this information with the known distance between the retransmission modules 161a-161c, the location of the bolus 10 can be determined. Alternately, either the directional 184 or omnidirectional antenna 182 may be used to transmit the data. The device may switch between the antennas 184 and 182 based on operational needs such as general reception of data, and location of a particular bolus. The retransmitter modules 161a-161c choice of antenna can be determined directly by the retransmission modules 161a-161c, remotely by the base station 186, or by the bolus 10.

The retransmitter module 185 will receive the bolus data at a particular frequency, perform preprocessing as required, and transmit the data to the base station 186 at a higher frequency. The bolus 10 may contain memory to store data until such time as it is ready for transmission to the base station 186. Control circuitry and software to perform required data processing and manage transmission of data to the base station, as the base station is available to receive the data.

The retransmitter module 185 may draw power directly from an existing power source such as existing 120V/240V circuits, may utilize a photovoltaic array or wind turbines to charge a battery, or some combination of both. It may also have a backup battery or other uninterruptable power source to continuously supply power to the unit, in the event of a temporary loss of power.

The retransmitter module 185 may be located at a fixed point such as a feed bunk or watering trough or may move in a random or predetermined path. If the device is not at a fixed location, it may continuously charge its power cell, as previously described, or return to a predetermined location to recharge the power cell. A non-stationary device may traverse the area of interest above ground using wires or some other means and may draw power from this means or a wire above, for example. Alternately, it may traverse the area of interest utilizing a mobile platform that has wheels, tracks, or other suitable means of locomotion.

The base station 186 shown in FIG. 10 includes a transceiver 188, an antenna 187, computing device 189, and a communication component 190 capable of accessing the internet. The transceiver 188 may be used for receiving data from the retransmitter module 185 and sending commands to the retransmitter module 185 for use by either the base station 186 or a particular bolus. In certain instances, the retransmitter modules 185 may act as relays for other retransmitter modules 185, both receiving data and transmitting commands. The computing device 189 includes a monitor, central processing unit, keyboard, and other related peripherals typically associated with a computing device capable of performing the operations described herein. The computing device may be a desktop computer, a laptop computer, a tablet, a PDA or a mobile device such as a smart phone. The communication component 190 may be a hardwired system such as a T3 line or DSL connection or may be a wireless connection such as a fourth generation (4G) wireless data transmitter.

The health monitoring bolus 10, when installed in the animal, will transmit data to a recipient such as the retransmitter module 185 described herein that may, in turn, transmit the data to a base station such as the base station 186 as described herein. The bolus 10 may process the data and send the final results to the base station 186 via the retransmitter module 185. Alternately, the bolus 10 may send raw signal data for analysis to be performed at the retransmitter module 185 or the base station 186. In this instance the bolus 10 may perform minor preprocessing to prepare the data for transmission to the retransmitter 185 or base station 186.

Also, when the transducer element 311 generates voltages because of the subject's motion, for example, the control circuitry 173 periodically routes this charge signal for charging the power cell 177, which provides power for the various components of the bolus 10.

As part of completing the fabrication of the bolus 10, the sensor module 11 and the control and data transmission module 19 are sealed and tested for mechanical and electrical performance. Units that perform adequately are integrated with the fluid analysis module 15 using high volume, low cost assembly techniques. Upon completion of service, the units can be disassembled, tested, refurbished, sterilized and reintroduced into the manufacturing process.

The proposed assembly methods are facilitated by the use of mechanical features such as the threads, snap fits, mechanical stops, etc., previously described.

Table 1 provides data on the typical concentration of volatile fatty acids in rumen fluid and associated physical properties. These acids are considered weak acids since they partially disassociate in water (eq. 1).

TABLE 1
Typical concentrations of rumen fluid
(based on nominal value of 100 mmol/L)

| Acid | Concentration | pKa | Density (kg/m3) |
|---|---|---|---|
| Acetic Acid | 63.0% | 4.76 | 1049 |
| Propionic Acid | 20.7% | 4.87 | 990.00 |
| Butyric Acid | 13.5% | 4.82 | 959.50 |
| Valeric Acid | 1.1% | 4.82 | 930.00 |
| Isovaleric Acid | 1.2% | 4.86 | 969.70 |
| Isobutyric Acid | 0.6% | 4.78 | 925.00 |

HA+H₂O [symbol] H₃O⁺A⁻ (1)

Several analytical, numerical and statistical methods exist to calculate the pH of mixtures of weak acids and bases. One such equation for calculating the pH of weak acids under specific conditions is shown in equation 2 where pH is the value of the Hydrogen ion concentration and in a mixture of acids the summation of the concentration of the contribution of the individual Hydrogen ion concentrations (equation 3).

$$\begin{matrix}pH = log ( [ {H}_{3} {O}^{+} ] & ( 2 ) \\ pH = log ( {\sum }_{1}^{n} {[ {H}_{3} {O}^{+} ]}_{n} ) & ( 3 )\end{matrix}$$

Equation 4 represents the generalized solution of equation 2 where F is the concentration and x is the Hydrogen ion concentration. This equation can be rearranged into a second order polynomial and solved using the quadratic equation.

$${K}_{a} = \frac{[ {H}^{+} ] [ {A}^{-} ]}{[ H A ]} = \frac{{x}^{2}}{F - x} \qquad ( 4 )$$

If the concentrations of the individual acids are known, then using tabulated values for acid dissociation constant, Ka (pKa=−log(Ka), the concentration of Hydrogen ions can be determined (equation 5 under specific conditions and in a more general form using equation 4) and used to calculate pH (equation 3).

[H₃O⁺]=\sqrt{Kₐcₐ} (5)

In addition to weak acids, bases in the rumen fluid (e.g., ammonia, bicarbonate bases compounds from saliva, etc.) also contribute to the pH. To calculate the impact on pH of the rumen fluid, a similar method to the above for weak bases can be used. Equation 6 is typically used in the calculation of the pH. In this instance pH=14−pOH.

pOH=−Log₁₀[OH⁻] (6)

To determine pOH, equation 7 can be used to find the concentration (x) of OH⁻ by rearranging the equation into a second order polynomial and solving the equation using quadratic formula and tabulated values of Kb.

$${K}_{b} = \frac{[ O {H}^{-} ] [ {A}^{+} ]}{[ H A ]} = \frac{{x}^{2}}{F - x} \qquad ( 7 )$$

Using the above equations, solving for Hydrogen ions of various constituents the pH can be determined by a variety of analytical, statistical, and numerical models. FIG. 12 provides an overview of this process.

The bolus 10 in active mode sends a pulse of a form similar to that shown in FIG. 5 and receives a pulse similar to that shown in FIG. 13. With reference to FIG. 14, the time difference between transmit pulse 101 and received pulse 102 divided by the path length, which is twice the distance from the front face of the transducer element 311 of FIG. 3 and the front face of the reflector 47 of FIG. 4 can be used to calculate the speed of sound in a fluid. The drive pulse shown in FIG. 5 should have sufficient duration and rise time to generate a broad band pulse capable of exciting the transducer element 311 of the sensor module 11 at the resonant frequency of the transducer element 311 or at an “off resonance” frequency as required for analysis. The transducer element 311 is selected to be sufficiently broad band enough so that the received pulse has spectral content at the relaxation frequencies of the constituents of interest in the fluid under analysis/monitoring with the bolus 10.

The fluid acts on the pulse to reduce its amplitude and modify its frequency content. Analysis of the spectral content of the frequency both at the principle and higher harmonics of the received signal can be used to determine the concentration of various components of the rumen fluid including VFA content, dissolved gas, proteins, and other relevant constituents.

FIG. 15 shows the frequency spectrum of a 2.25 MHz immersion transducer placed in water and driven by an Olympus 5072PR pulser-receiver unit. This unit drives the transducer element 311, receives the return signal, conditions the signal and outputs it to a digital storage oscilloscope. The unit also provides a trigger signal to synchronize a display of both the transmitted and the received signals. Since rumen fluid is mostly water, this spectrum can be used as reference for determining concentrations of the various constituents. This spectrum was obtained by performing a Fast Fourier Transform (FFT) on the received time domain signal and can be subtracted from rumen fluid spectra to determine the concentration of a particular component. FIGS. 16 and 17 illustrate this concept for multiple concentrations of acetic acid.

FIGS. 16 and 17 were generated by collecting spectra from six different measurements on the fluid of interest on a digital storage oscilloscope. These measurements are combined in a single data file using PeakFit software and this program generated a curve fit for the data. This was done for all concentrations and a baseline of water. An inflection as a function of increasing Acetic acid concentration can be seen at approximately 1.8 MHz.

FIG. 18 shows the results of the baseline being subtracted from each spectra as well as secondary processing including curve fitting, smoothing, and filtering. Using the content of FIG. 14, and selecting the value at 1.8 MHz for the corrected amplitude, a calibration curve can be constructed as shown in FIG. 19. This curve is established by generating mixtures of water and various levels of Acetic acid and obtaining the frequency spectra at a constant temperature. The change in magnitude of the inflection as a result of the Acetic acid is plotted against the change in concentration and a regression model is fitted to the data to evaluate the goodness of fit and create a calibration curve.

This process can be repeated for the various constituents of rumen fluid at various temperatures to determine their concentrations. These values can be used directly or in conjunction with various analytical (such as those described above), statistical, and numerical methods to make predictions about the nature of the rumen fluid and ultimately the health and metabolic (production) efficiency of a particular animal or the herd in general.

The speed of sound of most fluids, including rumen fluid, is determined by the temperature, density, and bulk modulus of the fluid. The composite speed of sound is a function of the constituents of the fluids and their relevant physical properties. The relationship between speed (c), density (ρ) and bulk modulus (K) of either a composite fluid or its constituent fluids is represented in equation 8. Density is a function of temperature and impacts the speed of sound accordingly, i.e., increasing temperature equals a decrease in density resulting in a larger speed of sound.

$$c = \sqrt{\frac{K}{ρ}} \qquad ( 8 )$$

Based on reported values of bulk modulus for water, the primary constituent of rumen fluid, the change in bulk modulus over the expected temperature range of a cow is less than 0.5%. In practice this means that the speed of sound is primarily a function of the change in density of the liquid. This allows one to make predictions on the density of the rumen fluid using a secondary temperature sensor or prediction on the fluid's temperature using the speed of sound. Calibrations curves, similar to the ones described for the spectral method, can be created to characterize the various relationships between speed of sound and the properties of the rumen fluid. This information, when combined with the spectral data, allows a complete in-vivo characterization of the rumen fluid.

The fully analyzed data from a specific individual bolus of an animal under observation as well as from other devices in the herd can be used to diagnose the physiological and metabolic state of the particular animal and of the herd. This can be done via the base station 186 or remotely by a veterinary professional, nutritionist or other qualified party. This form of telemedicine allows analysis of animals and herds in various locations and the compilation of a profile of the health of animals in a multi-feedlot operation or at the national or international level. This information can be used by multiple parties both involved in the care and feeding of animals as well as agencies involved in the regulation of animal welfare and export.

The previously described analysis can be performed at the base station 186 or at a remote location. The data can be stored in physical storage devices or in a cloud based system. Storage on a cloud based system allows for the combination of data from various sets for a comprehensive analysis program as previously described.

The present invention has been described with respect to specific examples and particular usages. It is to be understood that various modifications may be made to the devices and methods described herein without departing from the spirit and scope of the invention. Accordingly, other embodiments are within the scope of the claims appended hereto.

What is claimed is:

1. A device for analyzing characteristics of a fluid, wherein the device is arranged to be located within the fluid, the device comprising:
a. a sensor module including a transducer element configured to convert sounds associated with the fluid into electrical signals;
b. a fluid analysis module configured to analyze the characteristics of the fluid based on information output by the sensor module;
c. a fluid pumping module configured to move the fluid with respect to the sensor module, wherein the fluid analysis module is connected to the fluid pumping module; and
d. a control and data transmission module electrically coupled to the fluid analysis module, wherein the control and data transmission module is configured to output information from the fluid analysis module to a receiver.

2. The device of claim 1 wherein the transducer element includes a piezoelectric element.

3. The device of claim 2 wherein the transducer element includes an acoustic lens with a profile arranged to focus an acoustic beam.

4. The device of claim 1 wherein the fluid is in a rumen or reticulum of an animal.

5. The device of claim 1 wherein the receiver is spaced away from the device.

6. The device of claim 5 wherein the receiver is a retransmitter.

7. The device of claim 6 wherein the retransmitter is mobile.

8. The device of claim 5 wherein the receiver is a base station.

9. The device of claim 1 wherein the output of information from the device may be combined with the output of other fluid analysis devices to aid in the assessment of the conditions of multiple sources of fluid.

10. The device of claim 9 wherein the information is output to a cloud computing system.

11. The device of claim 1 wherein the fluid is located in a tank of an industrial facility.

12. The device of claim 1 wherein one or more of the sensor module, the fluid analysis module, the fluid pumping module and the control and data transmission module is fabricated to enable refurbishment or replacement thereof so that the device can be reused after removal from the fluid.

13. The device of claim 1 wherein the control and data transmission module includes a charging circuit arranged to store voltage generated by one or more other components of the device and to supply power to one or more other modules of the device.

14. The device of claim 13 wherein the fluid pumping module includes one or more magnets, wherein the one or more magnets move with movement of the pumping module.

15. The device of claim 14 wherein the transducer element of the sensor module is arranged to generate the voltage stored by the charging circuit based on one or more of:
a. reception of vibrations from the fluid; and
b. induced electric fields generated by movement of the one or more magnets.

16. A method of analyzing one or more characteristics of a fluid using a device that may be located within the fluid, the method comprising the steps of:
a. establishing a baseline of signal spectra associated with one or more constituents of the fluid;
b. sending an acoustic signal transmission through the fluid;
c. receiving a responsive acoustic signal from the acoustic signal transmission, wherein the responsive acoustic signal represents characteristics of the fluid through which the acoustic signal passes;
d. generating spectra of the responsive acoustic signal;
e. comparing the generated spectra with the baseline spectra; and
f. outputting information about one or more constituents or one or more conditions of the fluid based on the comparison.

17. The method of claim 16 wherein the device includes a transducer element that sends the acoustic signal transmission through the fluid and receives the responsive acoustic signal.

18. The method of claim 16 wherein the fluid is in the rumen or reticulum of a ruminating animal and the one or more constituents or the one or more conditions is selected from the group consisting of VFA content, buffering compounds, ammonia, dissolved gas, proteins, pH, density and temperature.

19. The method of claim 16 further comprising the step of transmitting the outputted information to a remote facility.

20. The method of claim 19 wherein the fluid is located within an animal and the remote facility to where the information is outputted is an animal healthcare facility.

21. The method of claim 16 further comprising the step of sending a plurality of acoustic signal transmissions at predetermined or selectable intervals.

22. The method of claim 22 wherein the intervals are selected based on optimizing a balance between power management and generating enough readings to provide sufficient fidelity to accurately characterize the one or more constituents or one or more conditions of the fluid.

23. The method of claim 22 wherein the intervals may be adjusted.
